(19) United States
(12) Patent Application Publication
(10) Pub. No.: US 2026/0284134 A1
(43) Pub. Date: Sep. 24, 2026

(54) POLYMER-BASED HEMOSTATIC FORMULATIONS PROVIDED WITH ANTI MICROBIAL, ANTI-VIRAL AND ANTI-INFLAMMATORY PROPERTIES AND PRODUCTION METHOD THEREOF

(71) Applicant: Fikrettin SAHIN, Istanbul (TR)

(72) Inventors: Fikrettin SAHIN, Istanbul (TR); Sevda DEMIR, Istanbul (TR); Seda TOMAN, Istanbul (TR); Melis KALKAN, Istanbul (TR)

(73) Assignee: Fikrettin SAHIN, Istanbul (TR)

(21) Appl. No.: 19/135,822

(22) PCT Filed: Dec. 5, 2022
(86) PCT No.: PCT/TR2022/051412

§ 371 Date: Jun. 5, 2025

(51) Int. Cl. A61K 36/53; A61K 9/06; A61K 47/36; A61P 7/04; A61P 31/04; A61P 31/14; A61P 31/20

(52) U.S. Cl. CPC A61K 36/53; A61K 9/06; A61K 47/36; A61P 7/04; A61P 31/04; A61P 31/14; A61P 31/20

(57) ABSTRACT

Provided is a production method for gel formulations with hemostatic (bleeding stopping) effect, the gel formulations provide anti-microbial, anti-viral, anti-inflammatory effects and contain lavender (Lavandula spp.) oil. Formulations that can be safely used in the treatment of minor bleeding injuries, emergency response situations, surgical cuts and military injuries and burns are developed.

### CROSS REFERENCE TO THE RELATED APPLICATIONS

This application is the national phase entry of International Application No. PCT/TR2022/051412, filed on Dec. 5, 2022, the entire contents of which are incorporated herein by reference.

### TECHNICAL FIELD

The present invention relates to polymer-based formulations (Coagulant gel and Hemostat) with hemostatic (bleeding stopping) effect, which provide anti-microbial (antibacterial, antifungal, anticandidal), anti-viral, anti-inflammatory activity and contain lavender oil (Lavandula spp.).

### BACKGROUND

Uncontrolled bleeding and wound infection are one of the leading causes of death in medical wound care. Hemostasis is the stopping of bleeding by coagulation or physical pressure. Due to its clotting properties, it is also considered as the first stage of wound healing. In order to prevent blood clots from forming, endothelial cells of intact blood vessels prevent blood clotting with a heparin-like molecule and thrombomodulin and prevent platelet aggregation with nitric oxide and prostacyclin. When the endothelium of a blood vessel is damaged, the endothelial cells stop secretion of coagulation and aggregation inhibitors and instead secrete von Willebrand factor, which initiates the maintenance of hemostasis after injury. Hemostasis is achieved by the coordinated action of three different mechanisms: vasoconstriction, platelet plug formation and blood clotting (fibrin formation from inactive fibrinogen proteins) [1].

If the body's own mechanisms are not effective enough to stop the bleeding, it is necessary to apply additional hemostatic agents. Currently, surgeons use different methods to achieve rapid hemostasis. Physical methods such as manual compression and suturing may not be completely effective. Thermal methods such as laser, bipolar coagulation, and ultrasonic dissectors may cause serious complications connected with the development of infection and necrosis formation [2].

Hemostats, sealants and adhesives are used during surgical procedures to achieve hemostasis. Hemostatic agents vary widely related to their mechanism of action, composition, ease of application, adherence to tissue, immunogenicity and cost. These agents contribute to rapid hemostasis, better visualization of the surgical area, shorter operative times, decreased requirement for transfusions, decreased wound healing time and overall improvement in patient recovery time. While some antihemorrhagic systemic drugs used in medicine inhibit fibrinolysis or promote coagulation, locally acting hemostatic agents work by causing vasoconstriction or promoting platelet aggregation [3].

A wide range of traditional materials, usually based on cellulose, collagen and gelatin, are used for hemostasis applications [4]. However, in recent years, active coagulation agents (chitosan or kaolin) have been adapted to hemostatic materials to accelerate the blood coagulation process and reduce bleeding [5]. In particular, many new fabrics loaded with modified sodium starch glycolate, chitosan hydroquinone-based gauze or other hemostatic agents are being introduced [2,6]. The use of inorganic and organic nanofiber materials carrying other materials with mineral content or metal ion chelated tannic acid coatings, anti-microbial and anti-inflammatory agents has become widespread [7]. Essential oils derived from certain plants with hydrophobic, pain-relieving, anti-inflammatory, anti-microbial and anti-viral properties have been used to treat pain and bleeding due to dental infections in humans [8].

Essential oils are widely used in traditional and alternative medicine for their anesthetic, antiseptic, astrengenic, hemostatic, deodorant, diaphoretic, disinfectant, expectorant, febrifuge, fumigant, inhalant, insect repellent/preventive, sedative and stimulant and anti-scar, abscess, arthritis, asthma, boils, bronchitis, burns, cancer, diabetes, diarrhea, diphtheria, dysentery, encephalitis, enteritis, erysipelas, fever, influenza, inflammation, laryngalgia, laryngitis, leprosy, malaria, mastitis, miasma, pharyngitis, phytitis, rhinitis, wounds, sore throat, spasms, trachalgia. As the effectiveness of essential oils is scientifically proven especially with the studies conducted in the recent years, they are in high demand in many areas from cleaning living spaces to the medical and cosmetic sector [9]. Among these oils, Lavender oil has been scientifically shown to be effective in many different areas for many years and has been used in many different areas. In particular, it has been used topically, orally and by inhalation in many areas such as depression, anxiety, dementia, cardiovascular activity, cancer chemoprevention, anti-microbial activity, and aromatherapy. In vitro studies have shown antibacterial effects of lavender oil, especially against gram-negative microorganisms [10]. Inhibition of the growth of methicillin-sensitive and -resistant Staphylococcus aureus (MSSA and MRSA) was observed with the combined use of various lavender oils and other essential oils (e.g. Lavandula latifolia, Lavandula stoechas) [11]. In other studies, strong and rapid antiparasitic effects against Giardia duodenalis and Trichomonas vaginalis, as well as both fungistatic and fungicidal effects against Candida albicans [12, 13] were reported. After bacterial infections, it takes time for aphthous ulcers to heal and the area to repair. In a study on rabbits, it was reported that 90% of the animals with induced recurrent aphthous ulceration treated with lavender oil showed complete healing on day 3, while redness, ulcers and inflammation persisted in the placebo group. In the histological examinations of the same study, it was reported that while edematous and heavy infiltration of acute and chronic inflammatory cells was observed in the placebo group, the tissue had a near-normal appearance in the group with topical lavender oil application. In a double-blind clinical study on 115 patients with recurrent aphthous ulceration, it was found that 100% of the patients who applied 2% lavender gel topically showed improvement on Day 4, while the placebo group continued to have ulceration and pain [14].

The mechanisms of anti-viral activity of essential oils have been reported in the literature as capsid fragmentation, viral expansion, inhibition of virus entry into host cells due to the degradation of hemagglutinin protein in some viruses, or the ability of essential oils and their components to inhibit late stages of the viral life cycle by targeting the redox signaling pathway [15]. When lavender oil is evaluated in terms of anti-viral activity, research has shown its activity against herpes simplex type 1 and 2, Influenza H1N1 and H5N1 viruses [16, 17]. Lavender oil has become a popular candidate for the pharmaceutical and medical sector due to its biological activity demonstrated by in-vitro, in-vivo and clinical studies. For this reason, a wide range of products containing lavender oil, water and active phenolics have been introduced to the market in recent years. However, in the scientific studies conducted in the literature, many scientific articles have been published on the anti-inflammatory, anti-microbial and anti-viral activities of plant essential oils derived from lavender species. However, the hemostatic agent properties of lavender species oils have not been known so far. Within the scope of this study, it was determined for the first time that lavender oils are hemostatic agents, and it was planned to develop lavender oil-containing coagulant gel and hemostatic formulations and/or wound dressing materials with anti-microbial (antibacterial, antifungal, anticandidal), anti-viral and anti-inflammatory properties.

Although Lavandula oil is applied topically, orally and by inhalation for different purposes, it has not been utilized so far in terms of its coagulation properties. In the state-of-the-art, coagulant materials can contain organic and inorganic polymers, nanoparticles or nanofibers. Therefore, they can cause infections and allergic reactions in the areas where they are applied. Depending on the severity of infection and the area of application, septic shock may develop due to the use of hemostat. In addition, in the state-of-the-art, the fact that different materials are used for surgical injuries and superficial injuries is economically disadvantageous.

CN103446619, DE102009023269, RU2002121797 are some patent applications known in the art.

### SUMMARY

The objective of the invention is to develop polymer-based hemostatic formulations containing lavender oil with anti-microbial (antibacterial, antifungal, anticandidal), anti-viral and anti-inflammatory activity.

Another objective of the invention is to develop formulations in the form of gel and/or chitosan powder with lavender oil.

A further objective of the invention is to develop formulations that can be safely used in the treatment of minor bleeding injuries, emergency response situations, surgical cuts and military injuries and burns.

### DETAILED DESCRIPTION OF THE EMBODIMENTS

The invention relates to developing formulations with hemostatic effect including lavender oil having anti-microbial (antibacterial, antifungal, anticandidal), anti-viral, and anti-inflammatory activity in combination with gelling polymer to obtain gel form and/or in combination with chitosan content that enhances the aforementioned biological activities to obtain powder form. The formulations developed within the scope of the invention demonstrate the hemostatic effect of lavender oil on the one hand and exhibit antibacterial, antifungal, anticandidal, anti-viral and anti-inflammatory activity on the other hand, unlike the commercially available hemostats. The formulations of the present invention have all-natural ingredients and, with their biological activity, are able to prevent bacterial, fungal and viral infections that may occur in open bleeding. In addition, these formulations will have the potential to be used in the treatment of many autoimmune and allergic diseases thanks to their anti-inflammatory properties. These formulations developed within the scope of the invention can be safely used in the treatment of minor bleeding injuries, emergency response situations, surgical cuts and military injuries and burns, thanks to their biological activity, and hemostatic and hydrophobic properties. Thanks to these properties the product exhibits anti-microbial and anti-inflammatory properties while exhibiting blood stopping properties due to its hydrophobic and hemostatic structure.

The production method of the gel formulations with hemostatic effect containing lavender oil of the present invention includes the following process steps:
producing coagulant gel,
   adding lipogel slowly into lavender oil in a ratio of 1:1 by weight,
   stirring the resulting mixture at a stirring speed of 250 rpm until it is completely gelled,
   obtaining the coagulant gel (lipogel),
producing the hemostat formulation,
   dissolving polymerized chitosan (Sigma, 48165) in 1% (v/v) acetic acid to form a 4% (w/v) solution with a stirrer and turning it into gel form,
   mixing the coagulant gel formulation and the chitosan solution in a 1:2 ratio and turning it into liquid form,
   after allowing the prepared mixtures to rest at a temperature of −80-−96° C. for 24 hours, removing all the water therein with a lyophilizer at a temperature of −82-−96° C. with a vacuum power of 0.016 mBar,
   obtaining the hemostat formulation of the present invention.

The content of these formulations is as follows:
3. Oil gelling agent and lavender oil
4. Chitosan, oil gelling agent and lavender oil.

In one embodiment of the invention, the gel formulation with hemostatic effect including lavender oil contains 20%-50% lavender oil, 20%-50% oil gelling agent, for 1%-10% chitosan by volume.

The prepared formulations have antiseptic and hemostatic properties with anti-inflammatory, antibacterial, antifungal, anticandidal and anti-viral activity. These formulations can be in the form of lotions, creams, emulsions, sprays, ampoules, foams, gelatins, pastes, powders, and biomedical material applications containing them, or a combination thereof. In addition, hemostatic formulations with anti-inflammatory, antibacterial, antifungal, anticandidal and anti-viral activity can also be used by being loaded on different carrier materials. Within these product groups, product types such as gauze, bandages, sponges, tapes, wound dressings, wound closures or any combination thereof can be produced for blood stopping, and burn and wound healing purposes.

Both formulations created with lipogel and chitosan are completely different from existing patents in terms of content and are produced by providing new biological properties. The formulations are not only anti-inflammatory and antibacterial, but also exhibit anti-microbial and anti-viral activities. The obtained experimental results are given in Table 2, 3, and 4. The formulations do not pose a risk of embolism due to their ability to coagulate rapidly and form gelation in layers, allowing them to be used successfully in all types of bleeding. Therefore, it can be used not only against damage and injuries specific to one part of the body, but also against all internal and external damage, injury and bleeding, such as surgical interventions, war wounds, oral injuries and operations.

### Experimental Studies

### Production of Coagulant Gel (Lipogel) Formulation:

In order to produce 1000 mg of coagulant gel, 500 mg of Lipogelac is added slowly into 500 mg of lavender oil. The mixture is stirred at 250 rpm until completely gelled.

### Production of Hemostat Formulation

Preparation of polymer solution: The polymer (chitosan) powder was dissolved in a distilled water solution with 1% (v/v) acetic acid at a ratio of 4% (w/v) and turned into gel form, respectively. The coagulant gel formulation and chitosan solution were mixed with acetic acid and dH20 in a 1:2 ratio and turned into a liquid form. Due to the carrier properties of the substances, it should be done in a 1:2 ratio for maximum efficiency. The prepared mixtures were allowed to rest at −80° C. for 24 hours and then all the water therein was removed with a lyophilizer (Ilshine Biobase) at −82° C. with a vacuum power of 0.016 mBar.

### Anti-Microbial Experiments

Antibacterial and antifungal tests for the prepared formulations were analyzed by international standard methods. For antibacterial tests, EN13727 (Chemical disinfectants and antiseptics-Quantitative suspension test for the evaluation of bactericidal activity in the medical area) and for antifungal tests, EN13624 (Chemical disinfectants and antiseptics-Quantitative suspension test for the evaluation of fungicidal or yeasticidal activity in the medical area) were used. In summary, following the dilution of the test sample, the products are introduced into a specific bacterial/fungal suspension. After 2 minutes of contact time, the mixture is neutralized and inoculated into the appropriate medium to calculate microorganism reduction. After the incubation period, viability is assessed.

### Anti-Viral Experiments

Cytotoxicity Assay: The effect of the prepared compounds on cell viability was measured by a substance called MTS based on mitochondrial dehydrogenase enzyme activity. In this method, HaCaT (immortalized human keratinocyte cells) were prepared in medium and seeded in 96-well culture plates with 5000 cells per well. After the incubation period (24 hours), the medium on the cell was removed and the compounds at certain concentrations were diluted with medium and applied on the cell. The response of the cells to the toxicity of the molecules was detected by measuring cell viability after 72 hours. After the incubation period is completed, the MTS substance, which is added onto the cells together with the medium, causes formation of colored formazan crystals as an indicator of cell viability. This color change was evaluated based on the absorbance measurement by using ELISA plate reader. The obtained results were analyzed.

Calculation of Infectivity Titer with TCID 50

The cells were removed from the flask and transferred to a 15 ml falcon tube and centrifuged at 500×g for 5 minutes. The medium on the cells that precipitated on the bottom of the falcon tube was discarded and 1 ml of new medium was added thereon and dissolved by means of a pipette. Then, the cells were seeded in 96-well plates to be monolayer within 24±2 hours and incubated at 37° C. in a 5% CO₂ incubator. When the cells were observed to be monolayer under an inverted microscope, the cells were processed. Vero cell line was used for Adenovirus and Poliovirus, Raw cell line was used for Murine norovirus. The substances whose non-toxic doses were determined were prepared with virus medium according to the volume to be used.

225 μl of virus medium was added to a new 96-well plate and 25 μl of virus was added to the first 6 wells of the 96-well plate and serial dilutions were made on a logarithmic scale (Log 10). The medium of the prepared monolayered cells is discarded. They were washed twice with virus medium. Serial dilutions prepared in a new 96-well plate were transferred on the cells and the medium volume was completed to 200 μl with the prepared non-toxic dose of the substance. Plates were incubated in a 37° C. 5% CO₂ incubator for 72 hours. At the end of the incubation period, the cytopathic effect (CPE) due to virus suspension was evaluated under an inverted microscope. The obtained results were evaluated by performing TCID50 calculation with Spearman-Karber method according to the following formulas.

$$M = {X}_{k} + d [ 0.5 - ( \frac{1}{n} ) ( r ) ] \qquad ( Formula 1 )$$

wherein:
M: Anti-viral activity value.
Xₖ: The highest dilution dose.
d: difference between dilutions.
n: number of wells per dilution.
r: sum of (−) responses.

$${M}_{V} = lg ( \frac{{V}_{a}}{{V}_{c}} ) = lg ( {V}_{a} ) - lg ( {V}_{c} ) \qquad ( Formula 2 )$$

wherein:
Mv: anti-viral activity value.
1 g (Vₐ): logarithmic mean of two biological replicates for control tests.
1 g (V_{c}): logarithmic mean of two biological replicates for the experimental groups.

Formula 1 and Formula 2 were used to calculate the anti-viral activities in Table 3.

In the European Union (EN14476, EN17272 etc.) standards on anti-viral activity studies, it is stated that in order to be able to state that a product has an anti-viral activity, a 4 log reduction should be achieved on the viruses used in the experiment. In this context, anti-viral activity of 4 log and above was detected for Adenovirus Type 5, Poliovirus Type 3, Murine Norovirus, Vaccinia Virus, Bovine Coronavirus and Herpes Simplex Virus Type 1 in both formulations we have made (Tables 2 and 4).

### Coagulation Experiments

1000 cc of human venous blood was placed in a petri dish and the following amounts of substances were added thereto. Separate time measurements were taken for each substance experiment.

TABLE 1
Substance names and amounts for in-vitro coagulation experiments.

| Substance Name | Amount (mg or cc) |
|---|---|
| Lipogel (Lipogelac) | 450 |
| Chitosan | 450 |
| Lavandula Oil | 450 |
| Blood | 1000 |
| PBS | 450 |

### In-Vivo Experiment of Hemostat Formulation

Albino rats (9 weeks old) were incised in the femoral artery, femoral vein, renal artery and liver and blood flow was ensured. Immediately after the onset of blood flow, hemostat application was performed, and the duration was monitored.

The results obtained from the analysis of the formulations are as follows.

### Coagulant Gel Anti-Viral Activity Result

Anti-viral activity evaluation of the obtained formulations was performed against various DNA-RNA viruses and the results are shown in Table 2.

TABLE 2
Anti-viral activity values of Lavandula Gel

| Virus Name | Activity Value (Log₁₀) |
|---|---|
| Adenovirus Type 5 | 4.0 |
| Poliovirus Type 3 | >4.0 |
| Murine Norovirus | 4.0 |
| Vaccinia Virus | >4.0 |
| Bovine Coronavirus | 4.0 |
| Herpes Simplex Virus Type 1 | >5.0 |

### In-Vitro Experimental Results for Chitosan-Based Formulation

### Anti-Microbial Activity

TABLE 3
Anti-microbial activity values of Hemostat

Activity Value (Log₁₀)

| Microorganism Name | 10⁰ | 10⁻¹ | 10⁻² |
|---|---|---|---|
| Pseudomonas aeruginosa | >5.0 | >5.0 | <4.0 |
| Staphylococcus aureus | >5.0 | >5.0 | <4.0 |
| Enterococcus hirae | >5.0 | >5.0 | <4.0 |
| Klebsiella pneumoniae | >5.0 | >5.0 | <3.0 |
| Bacillus subtilis | >5.0 | >5.0 | <3.0 |
| Escherichia coli | >5.0 | >5.0 | <4.0 |
| Candida albicans | >4.0 | >4.0 | <2.0 |
| Aspergillus spp. | >4.0 | — | — |
| Penicilium spp. | >4.0 | >4.0 | <1.0 |
| Methicillin-resistant | >5.0 | — | — |
| Staphylococcus aureus | | | |
| Vancomycin-resistant | >5.0 | — | — |
| Enterococcus | | | |
| Acinetobacter spp | >5.0 | — | — |

### Anti-Viral Activity

TABLE 4
Anti-viral activity values of Hemostat.

| Virus Name | Activity Value (Log₁₀) |
|---|---|
| Adenovirus Type 5 | 4.0 |
| Poliovirus Type 3 | >4.0 |
| Murine Norovirus | 4.0 |
| Vaccinia Virus | >4.0 |
| Bovine Coronavirus | 4.0 |
| Herpes Simplex Virus Type 1 | >5.0 |

### Evaluation of Coagulation Formation

### Control Group

TABLE 5
Time-dependent evaluation of coagulation formation
of the formulation in control group

| Substance Name | Amount (mg or cc) | Time (h:min:sec) |
|---|---|---|
| Geloc | 450 | about 00:05:00 |
| Lavandula Oil | 450 | about 00:05:00 |
| Blood | 1000 | >00:08:00 |
| PBS | 450 | >00:08:00 |

### Experimental Group

TABLE 6
Time-dependent evaluation of coagulation
formation of the formulation

| | Amount Coagulant | Time |
|---|---|---|
| Formulation | (mg or μl) | (h:min:sec) |
| Coagulant Gel + 2% Chitosan | 450 | 00:00:60 |
| Coagulant Gel + 3% Chitosan | 450 | 00:00:30 |
| Coagulant Gel + 4% Chitosan | 450 | 00:00:23 |

In the studies, the maximum carrier ratio of chitosan was determined as 4%.

### In-Vivo Experimental Result for Chitosan-Based Formulation

The results obtained in femoral artery, femoral vein, renal artery and liver incisions in rats are as follows.

TABLE 7
Time-dependent evaluation of coagulation formation in femoral
artery, femoral vein, renal artery and liver incisions.

| Application Area | Amount (mg) | TIME (h:min:sec) |
|---|---|---|
| Femoral artery | 400 | 00:00:39 |
| Femoral vein | 400 | 00:01:03 |
| Renal artery | 400 | 00:00:33 |
| Liver | 400 | 00:00:38 |

### REFERENCES

[1]. Gil, Morayma Reyes. “Overview of the coagulation system.” Transfusion Medicine and Hemostasis. Elsevier, 2019. 559-564.
[2]. Suchý, Pavel, et al. “Composite hemostatic nonwoven textiles based on hyaluronic acid, cellulose, and etamsylate.” Materials 13.7 (2020): 1627.
[3]. Vyas, Krishna S., and Sibu P. Saha. “Comparison of hemostatic agents used in vascular surgery.” Expert opinion on biological therapy 13.12 (2013): 1663-1672.
[4]. Chiara, Osvaldo, et al. “A systematic review on the use of topical hemostats in trauma and emergency surgery.” BMC surgery 18.1 (2018): 1-20.
[5]. Hu, Zhang, et al. “Chitosan-based composite materials for prospective hemostatic applications.” Marine drugs 16.8 (2018): 273.
[6]. Panwar, Vineeta, et al. “In-vitro and in-vivo evaluation of modified sodium starch glycolate for exploring its haemostatic potential.” Carbohydrate Polymers 235 (2020): 115975.
[7]. Koopmann, Ann-Kathrin, et al. “Tannin-Based Hybrid Materials and Their Applications: A Review.” Molecules 25.21 (2020): 4910.
[8]. Edris, Amr E. “Pharmaceutical and therapeutic potentials of essential oils and their individual volatile constituents: a review.” Phytotherapy Research: An International Journal Devoted to Pharmacological and Toxicological Evaluation of Natural Product Derivatives 21.4 (2007): 308-323.
[9]. Bakkali, Fadil, et al. “Biological effects of essential oils-a review.” Food and chemical toxicology 46.2 (2008): 446-475.
[10]. Lodhia M H, Bhatt K R, Thaker V S. Antibacterial activity of essential oils from palmarosa, evening primrose, lavender and tuberose. Indian J Pharm Sci. 2009; 71 (2): 134-136.20336210
[11]. Roller S, Ernest N, Buckle J. The antimicrobial activity of high-necrodane and other lavender oils on methicillin-sensitive and -resistant Staphylococcus aureus (MSSA and MRSA). J Altern Complement Med. 2009; 15 (3): 275-279.19249919 Rouzet M. Lab Pharma Probl Tech. 1984; 32:462-466.
[12]. Moon T, Wilkinson J M, Cavanagh H M. Antiparasitic activity of two Lavandula essential oils against Giardia duodenalis, Trichomonas vaginalis and Hexamita inflata. Parasitol Res. 2006; 99 (6): 722-728.16741725
[13]. D'Auria F D, Tecca M, Strippoli V, Salvatore G, Battinelli L, Mazzanti G. Antifungal activity of Lavandula angustifolia essential oil against Candida albicans yeast and mycelial form. Med Mycol. 2005; 43 (5): 391-396.16178366
[14]. Altaei D T. Topical lavender oil for the treatment of recurrent aphthous ulceration. Am J Dent. 2012; 25 (1): 39-43.22558691
[15]. Wani, Abdul Rouf, et al. “An updated and comprehensive review of the antiviral potential of essential oils and their chemical constituents with special focus on their mechanism of action against various influenza and coronaviruses.” Microbial Pathogenesis 152 (2021): 104620.
[16]. Abou Baker, Doha H., et al. “Antiviral activity of Lavandula angustifolia L. and Salvia officinalis L. essential oils against avian influenza H5N1 virus.” Journal of Agriculture and Food Research 4 (2021): 100135.
[17]. Wińska, Katarzyna, et al. “Essential oils as antimicrobial agents-myth or real alternative?.” Molecules 24.11 (2019): 2130.

What is claimed is:

1. A gel formulation with hemostatic effect, comprising a lavender oil content containing an oil gelling agent and lavender oil.

2. A gel formulation with hemostatic effect, comprising a lavender oil content containing chitosan.

3. The gel formulation with hemostatic effect according to claim 2, wherein the gel formulation with hemostatic effect comprises 20%-50% of lavender oil, 20%-50% of oil gelling agent, and 1%-10% of the chitosan by volume.

4. The gel formulation with hemostatic effect according to claim 2, having a form at least one selected from a lotion, a cream, an emulsion, a spray, an ampoule, a foam, a gelatin, a paste, a powder, and a biomedical material application containing one of the lotion, the cream, the emulsion, the spray, the ampoule, the foam, the gelatin, the paste, and the powder.

5. The gel formulation with hemostatic effect according to claim 2, wherein the gel formulation with hemostatic effect is used by being loaded on a carrier material.

6. The gel formulation with hemostatic effect according to claim 5, wherein the carrier material is at least one of a gauze, a bandage, a sponge, a tape, a wound dressing, and a wound closure, and the gel formulation with hemostatic effect is loaded for blood stopping, burn and wound healing.

7. A production method of the gel formulation with hemostatic effect according to claim 2, comprising steps of:
producing coagulant gel,
adding lipogel to lavender oil to obtain a first mixture,
stirring the first mixture until the first mixture is completely gelled,
obtaining the coagulant gel,
producing a hemostat formulation,
dissolving polymerized chitosan in acetic acid with a stirrer to form a solution and turning the solution into gel form to obtain a chitosan gel,
mixing the coagulant gel with the chitosan gel to obtain a second mixture, and turning the second mixture into liquid form to obtain a resulting liquid,
after allowing the resulting liquid to rest, removing all water therein with a lyophilizer,
obtaining the hemostat formulation.

8. The production method according to claim 7, wherein the lipogel is slowly added to the lavender oil in a ratio of 1:1 by weight.

9. The production method according to claim 7, wherein the first mixture is stirred at a stirring speed of 250 rpm until completely gelled.

10. The production method according to claim 7, wherein the polymerized chitosan is dissolved in 1% (v/v) acetic acid to form a 4% (w/v) solution.

11. The production method according to claim 7, wherein the coagulant gel and the chitosan gel are mixed in a 1:2 ratio.

12. The production method according to claim 7, wherein the second mixture is allowed to rest at a temperature of −80° C. to −96° C. for 24 hours and then all the water therein is removed with the lyophilizer at a temperature of −82° C. to −96° C. with a vacuum power of 0.016 mBar.

13. The gel formulation with hemostatic effect according to claim 1, having a form at least one selected from a lotion, a cream, an emulsion, a spray, an ampoule, a foam, a gelatin, a paste, a powder, and a biomedical material application containing one of the lotion, the cream, the emulsion, the spray, the ampoule, the foam, the gelatin, the paste, and the powder.

14. The gel formulation with hemostatic effect according to claim 3, having a form at least one selected from a lotion, a cream, an emulsion, a spray, an ampoule, a foam, a gelatin, a paste, a powder, and a biomedical material application containing one of the lotion, the cream, the emulsion, the spray, the ampoule, the foam, the gelatin, the paste, and the powder.

15. The gel formulation with hemostatic effect according to claim 1, wherein the gel formulation with hemostatic effect is used by being loaded on a carrier material.

16. The gel formulation with hemostatic effect according to claim 3, wherein the gel formulation with hemostatic effect is used by being loaded on a carrier material.

17. The gel formulation with hemostatic effect according to claim 4, wherein the gel formulation with hemostatic effect is used by being loaded on a carrier material.

18. The production method according to claim 7, wherein the gel formulation with hemostatic effect comprises 20%-50% of the lavender oil, 20%-50% of oil gelling agent, and 1%-10% of the chitosan by volume.

19. The production method according to claim 7, wherein the gel formulation with hemostatic effect has a form at least one selected from a lotion, a cream, an emulsion, a spray, an ampoule, a foam, a gelatin, a paste, a powder, and a biomedical material application containing one of the lotion, the cream, the emulsion, the spray, the ampoule, the foam, the gelatin, the paste, and the powder.

20. The production method according to claim 7, wherein the gel formulation with hemostatic effect is used by being loaded on a carrier material.
